(19) United States
(12) Patent Application Publication
(10) Pub. No.: US 2009/0298946 A1
(43) Pub. Date: Dec. 3, 2009

(54) Local Anesthetic Deactivation

(76) Inventor: Cody R. Cleavelin, Lubbock, TX (US)

Correspondence Address: Roman Aguilera III, 9805 Beaufort Avenue, Lubbock, TX, 79424, US

(21) Appl. No.: 12/474,273

(22) Filed: May 29, 2009

Related U.S. Application Data
(60) Provisional application No. 61/130,673, filed on Jun. 2, 2008.

(51) Int. Cl. A61K 31/194; A61P 43/00

(52) U.S. Cl. 514/574

(57) ABSTRACT

A chemical solution for deactivating the effects of local anesthetic is disclosed. Local anesthetic is important in many medical procedures, such as dentistry, surgical procedures, and veterinary medicine. In many cases, after the procedure has been completed, the need for blocking the nerve conductance is no longer needed or wanted, and the effects of local anesthesia can last for several hours post procedure. This invention works by two mechanisms, manipulating the pH and calcium concentration of the local cellular environment. The invention can be delivered via an oral transmucosal delivery device or via an injectable. It provides a safe and biologically acceptable means to quickly eliminate the effects of local anesthetic. The use of this invention will allow patients to quickly gain back their normal nerve function.

### CROSS-REFERENCE TO RELATED APPLICATIONS

This application claims the benefit under Title 35 United States Code § 119(e) of U.S. Provisional Application No. 61/130,673; Filed: Jun. 2, 2008, the full disclosure of which is incorporated herein by reference.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention relates to the deactivation of local anesthetic in patients. More specifically, the present invention relates to a chemical solution used to deactivate the effects of local anesthetics. The solution may be administered by various delivery methods such as an injectable, a gel, a starch based film, a lozenge, or other transmucosal methods.

### BACKGROUND OF THE INVENTION

Without limiting the scope of the disclosed invention, the background is described in connection with a novel approach to the deactivation of local anesthetic.

Local anesthetic is important in many medical procedures, such as dentistry, surgical procedures, and veterinary medicine. In many cases, after the procedure has been completed, the need for blocking the nerve conductance is no longer needed or wanted, and the effects of local anesthesia can last for several hours post procedure. For example, the majority of dental procedures that employ local anesthesia do not have a need for pain management after the procedure has been completed. Prolonged blocking of nerve conductance is an inconvenience at the least and can be the cause of injury. Patients experience discomfort when trying to return to their normal daily tasks with the prolonged numbness. This prolonged numbness causes difficulty in eating, drinking, speaking, and can cause patients, especially children, to unknowingly chew the inside of their mouth or tongue. The use of this invention will allow patients to quickly gain back their normal nerve function.

### BRIEF SUMMARY OF THE INVENTION

All local anesthetics are weak bases and are classified as amino-esters or amino-amides. The structure of the local anesthetic molecule contains an aromatic ring and connected to that via an intermediate chain of either an amide or an ester is the amino group. These hydrophilic and hydrophobic components are what provide the local anesthetic qualities of being able to pass through the cell's lipid bilayer and also be soluble in the water environment outside and inside the cell. This invention will take these qualities and manipulate the immediate environment to stop the effects of the local anesthetic by drawing the molecule out of the cell.

Local anesthetics work to block nerve conduction by reducing the influx of sodium ions into the nerve cytoplasm. Sodium ions cannot flow into the neuron, and the potassium ions cannot flow out, thereby inhibiting the depolarization of the nerve. If this process can be inhibited for just a few Nodes of Ranvier along the way, then nerve impulses generated downstream from the blocked nodes cannot propagate to the ganglion. Local anesthetics bind directly to the intracellular voltage-dependent sodium channels therefore stabilizing the inactivated state of the sodium channels. This invention will reverse this process by safely manipulating the cellular environment drawing the local anesthetic out of the cell allowing the nerve to naturally regain its electrical potential gradient.

The present invention, therefore, provides a method and chemical composition to deactivate the effects of local anesthetic. The chemical solution is comprised of an acidic buffer coupled with a calcium salt and is administered by various delivery methods such as an injectable, a gel, a starch based film, a lozenge, or other transmucosal methods. This invention employs a novel approach to reversing quickly the effects of local anesthetic in a safe and biologically acceptable manner. This approach is distinct from other methods and solutions utilized. For example, most other solutions employ the use of a vasodilator for effectiveness in reversing the effects of local anesthetics. There are many documented adverse effects with the use of vasodilators. As such, the present invention has been developed to achieve equal effectiveness without the use of a vasodilator. In addition, the chemical composition is not limited to an injectable, but may be administered via various delivery methods. These various delivery methods allow for the tailoring to various patient groups that will be appreciated by one of ordinary skill in the art.

In summary, the present invention discloses an improved method and chemical composition to quickly eliminate the effects of local anesthetics. The use of this invention allows the patient to quickly gain back their normal nerve function. More specifically, by extension, the disclosed method and chemical composition can be used to treat animals as well.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which:

FIG. 1 is a diagram of how local anesthetic enters a cell at a biologically basic pH in accordance with embodiments of the disclosure;

FIG. 2 is a geometric representation of Citric Acid in accordance with embodiments of the disclosure;

FIG. 3 is a geometric representation of Calcium Citrate in accordance with embodiments of the disclosure.

### DETAILED DESCRIPTION OF THE INVENTION

Disclosed herein is a novel approach to deactivating local anesthetics in patients. The numerous innovative teachings of the present invention will be described with particular reference to several embodiments (by way of example, and not of limitation).

### pH Manipulation:

This invention will work by two mechanisms, manipulating the pH and calcium concentration of the local cellular environment. First looking at pH manipulation, local anesthetics are packaged as acidic salts but after injection it quickly takes on the pH of the surrounding tissue, and the molecular structure shifts between two forms; a dissociated molecule (RN) and an associated molecule (RNH+). These two forms of the anesthetic molecule exist in an equilibrium dependent upon the pKa of the anesthetic and the pH of the tissue. For example if the pH is lowered (acidic) the equation would shift to the left.

RNH⁺ [symbol] RN+H⁺

The Henderson-Hasselbach equation below shows that if the pH is below the pKa, the ratio will be <1. Therefore by lowering the pH in the area of the local anesthetic, a higher concentration of the associated molecule, RNH⁺, will occur. (A=RN, HA=RNH)

$$pH = {pK}_{a} = log \frac{[ {A}^{-} ]}{[ HA ]}$$

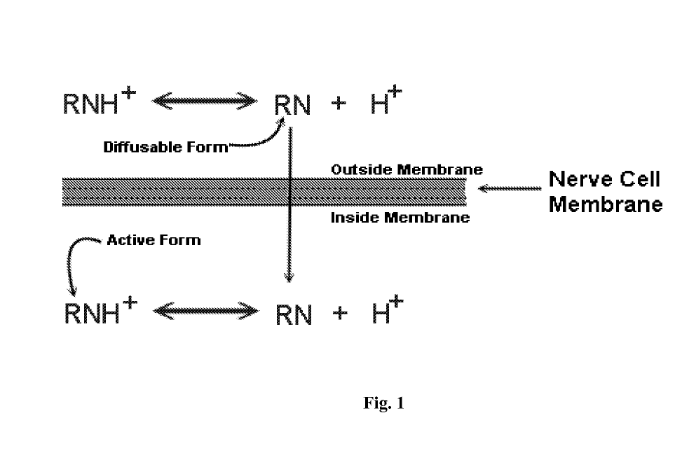

Reference is first made to FIG. 1, wherein a schematic is presented showing how local anesthetic enters a cell at a biologically basic pH. The majority of local anesthetics have a pKa of about 7 to 8. Therefore in the pH of the patient's tissue, the local anesthetic would have a higher percentage of the uncharged form which is on the right side of the equation. The uncharged form penetrates the membrane and then the charged form binds to a receptor site. Consistent with LeChâtlier's principle, creating an acidic environment would reverse the direction of the equation pulling the local anesthetic out of the cell, therefore creating a barrier not allowing the local anesthetic back into the cell.

### Calcium Concentration Manipulation: Deactivating Function of Local Anesthetic and Vasoconstrictor

The second mechanism, calcium concentration, provides for two benefits, hindering the function of both the local anesthetic and the function of the vasoconstrictor.

Transmission along the nerve pathway within the nerve sheath is accomplished by using sodium conductance. Local anesthetics work by hindering the sodium conductance and therefore block the nerve signal. Calcium improves local anesthetic function because it competes for the same phospholipid receptor as for the sodium. Reducing the available calcium ions from the cellular fluid, removing them from the nerve sheath would proportionately increase the available sodium; therefore allowing for sodium conductance along the nerve pathway. This invention uses a buffer containing a calcium salt which increases the external calcium concentration away from the nerve sheath.

Regarding the vasoconstriction aspect, it should be noted that the majority of local anesthetics contain a vasoconstrictor, usually epinephrine, to constrict the blood vessels. This decreases the rate of vascular absorption which allows more anesthetic to reach the nerve membrane and improves the depth of anesthesia. 1:200,000 volumetric units of epinephrine are common. Vasoconstrictors depend on cellular calcium ion concentrations to function. Using this invention will deactivate the vasoconstrictor by lowering the cellular calcium concentration. This is accomplished without the use of potentially hazardous vasodilators. This allows the area to return to a physiologic normal dilation which would increase blood flow to the local area. This would enable the body to naturally metabolize the local anesthetic in the liver and therefore be broken down and processed through the kidneys.

### Invention Component:

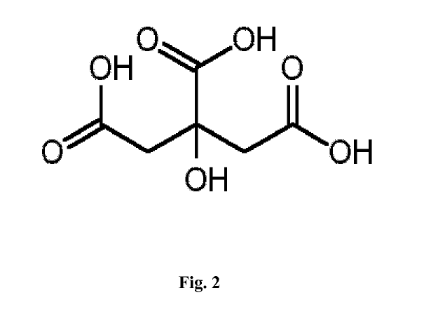

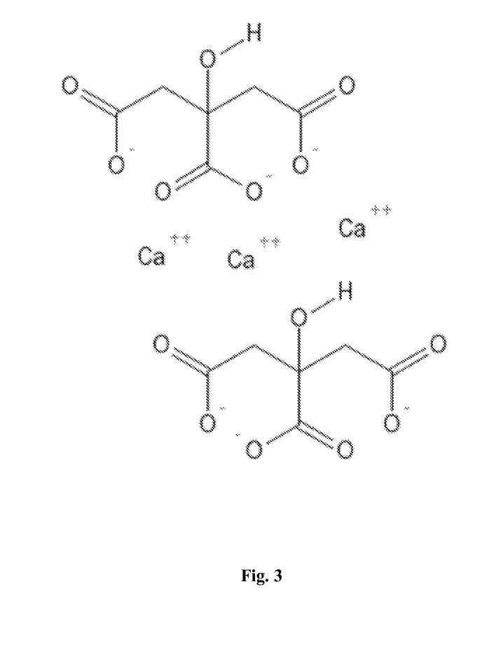

Reference is now made to FIGS. 2 and 3, wherein the geometric representations of Citric Acid and Calcium Citrate, respectively, are shown. As one specific example, the local anesthetic deactivator can be manufactured in the following way. At room temperature and ambient pressure using buffer manufacturing techniques commonly known in the art, stirring Citric Acid (FIG. 2), and Calcium Citrate (FIG. 3), in deionized water at a ratio of 4 to 1. This ratio will produce a solution with an appropriate calcium concentration and an approximate pH of 4. It is further noted that one determining factor for the effectiveness of the solution is the solution's pH. The target pH for maximum effectiveness of this invention is 4.7. However, in certain situations, a pH between 5 and 6 is desired so as to not reverse the effects of local anesthesia as quickly. For example, the reversal of the effects of local anesthetic is desired, but because of local inflammatory responses to a medical procedure, an expedited but not immediate reversal of the effects is desired.

With solutions such as this example, an invention will result that has the appropriate pH and calcium ion concentration to safely and quickly deactivate the function of a local anesthetic. By those skilled in the art it can be understood that this example can be administered via an oral transmucosal delivery system such as the following, but not limited to, a dissolving oral gel, a lozenge with a plastic handle, or a starch based dissolving film, all of which not requiring an injection. By way of example the oral dissolving gel can be comprised of a combination of glycols, such as propylene glycol and polyethylene glycol. Also known by those skilled in the art the invention can be administered by injection via a syringe. Lastly, the solution may be flavored by various means, such as but not limited to, a simple sugar, or a non-sugar sweetener.

The disclosed invention is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to describe specific embodiments of the invention, but their usage does not delimit the disclosed invention, except as may be outlined in the claims.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures and chemical composition described herein. Such equivalents are considered to be within the scope of this invention and are covered by the claims.

All publications and patent applications mentioned in the specification are indicative of the level of those skilled in the art to which this invention pertains. All publications and patent application are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the methods and/or chemical compositions disclosed and claimed herein can be made and executed without undue experimentation in light of the present disclosure. While the various aspects of this invention have been described in terms of preferred embodiments, it will be apparent to those skilled in the art that variations may be applied to the chemical composition and in the steps or in the sequence of steps of the method described herein without departing from the concept, spirit, and scope of the invention.

More specifically, it will be apparent that certain substances which are both functional and material related may be substituted for the substances described herein while the same or similar results would be achieved. All such similar substitutes and modifications apparent to those skilled in the art are deemed to be within the spirit, scope, and concept of the invention as defined by the appended claims.

### REFERENCES

1. Steven T. Blackman, Irene Ralske; Thames Pharmacal Co., Inc.: Gel Bases for Pharmaceutical Compositions. U.S. Pat. No. 4,883,660
2. John F. Butterworth IV, M.D., Gary R. Strichartz, Ph.D.: Molecular Mechanisms of Local Anesthesia: A Review. Anesthesiology 72:711-734, 1990
3. Elliot V. Hersh, DMD, MS, PhD, Paul A. Moore, DMD, PhD, MPH, Athena S. Papas, DMD, PhD, J. Max Goodson, DDS, PhD, Laura A. Navalta, BA, Siegfried Rogy, PhD, Bruce Rutherford, DDS, PhD, John A. Yagiela, DDS, PhD; AND the Soft Tissue Anesthesia Recovery Group: Reversal of Soft-Tissue Local Anesthesia With Phentolamine Mesylate in Adolescents and Adults. JADA 2008; Vol 139, No 8, 1080-1093
4. Bertil Hille: The pH-Dependent Rate of Action of Local Anesthetics on the Node of Ranvier. The Journal of General Physiology 69:475-496, 1977
5. Bertil Hille: Local Anesthetics: Hydrophilic and Hydrophobic Pathways for the Drug-Receptor Reaction. The Journal of General Physiology 69:497-515, 1977
6. Helen J. Kennedy and Roger C. Thomas: Effects of Injecting Calcium-Buffer Solutions on [Ca²⁺]_{I} in Voltage Clamped Snail Neurons. Biophys J 70:2120-2130 May 1996
7. Paul A. Moore, DMD, PhD, MPH, Elliot V. Hersh, DMD, MS, PhD, Athena S. Papas, DMD, PhD, J. Max Goodson, DDS, PhD, John A. Yagiela, DDS, PhD, Bruce Rutherford, DDS, PhD, Seigried Rogy, PhD, and Laura Navalta, M S: Pharmacokinetics of Lidocaine With Epinephrine Following Local Anesthesia Reversal With Phentolamine Mesylate. Anesthesia Progress Vol 55 Issue 2, pp. 40-48.
8. Pamela Palmer, Thomas Schreck, Stelios Tzannis, Andrew I. Poutiatine, and Larry Hamel: Drug formulations for oral transmucosal delivery to pediatric patients. USPTO Patent Application 20090010992.
9. Todd Scheuer: Commentary—A Revised View of Local Anesthetic Action: What Channel State Is Really Stabilized? J. Gen. Physiol. 113:3-6 1999
10. Shepley M P, Strichartz G R, Wang G K: Local Anesthetics Block non-inactivating Sodium Channels in a Use-dependent Manner in Amphibian Myelinated Axons. J Physiol (London) 341:62P, 1983
11. Strichartz G, Wang G K: The Kinetic basis for Phasic Local Anesthetic Blockade of neuronal Sodium Channels, Molecular and Cellular Mechanisms of Anesthetics. Edited by Roth S H, Miller K W. New York: Plenum Medical Book Co., 1986, pp. 217-226

1. A pharmaceutically safe solution for deactivating the function of local anesthetic which does not contain a vasodilator, comprising: an acidic buffer, calcium salt, and deionized water.

2. The solution of claim 1, wherein said solution has a pH of less than 6.

3. The solution of claim 1, wherein said solution has a pH of more than 3 and less than 6.

4. The solution of claim 1, wherein said solution has a pH of more than 5 and less than 6.

5. The solution of claim 1, wherein said solution has a pH of less than 6 and said calcium salt is calcium citrate.

6. The solution of claim 1, wherein said solution has a pH of more than 3 and less than 6, and said calcium salt is calcium citrate.

7. The solution of claim 1, wherein said solution has a pH of more than 5 and less than 6, and said calcium salt is calcium citrate.

8. The solution of claim 1, wherein said solution has a pH of less than 6, said acidic buffer is citric acid, and said calcium salt is calcium citrate.

9. The solution of claim 1, wherein said solution has a pH of more than 3 and less than 6, said acidic buffer is citric acid, and said calcium salt is calcium citrate.

10. The solution of claim 1, wherein said solution has a pH of more than 5 and less than 6, said acidic buffer is citric acid, and said calcium salt is calcium citrate.

11. The solution of claim 1, wherein said solution is an injectable.

12. The solution of claim 1, wherein said solution is not an injectable and is in a form allowing said solution to be delivered via an oral transmucosal method.

13. The solution of claim 12, wherein said transmucosal method is a gel.

14. The solution of claim 13, wherein said gel further comprises a gelling agent, an excipient, and a flavor.

15. The solution of claim 13, wherein said gel is comprised of a gelling agent at 1 to 10% by weight, an excipient at 1 to 20% by weight, said acidic buffer at 1 to 20% by weight, and total to 100% by weight with flavor and said deionized water.

16. The solution of claim 13, wherein said gel has glycol as a gelling agent and/or excipient.

17. The solution of claim 16, wherein said gel has polyethylene glycol and propylene glycol as a gelling agent and/or excipient.

18. The solution of claim 14, wherein said flavor to enhance taste comprises a simple sugar, or a non-sugar sweetener.

19. The solution of claim 12, wherein said transmucosal method is a lozenge.

20. The solution of claim 12, wherein said transmucosal method is a starched based dissolving film.

21. A method to reverse quickly the effects of local anesthetic in a safe and biologically acceptable manner, comprising the steps of: providing a pharmaceutically safe solution which does not contain a vasodilator, comprising an acidic buffer, calcium salt, and deionized water; determining the appropriate pH for desired effectiveness of said solution; and administering said solution to the patient via a delivery method.

22. The method of claim 21, further comprising the step of making said solution with a pH of less than 6.

23. The method of claim 21, further comprising the step of making said solution with a pH of more than 3 and less than 6.

24. The method of claim 21, further comprising the step of making said solution with a pH of more than 5 and less than 6.

25. The method of claim 21, further comprising the step of making said solution with a pH of less than 6 and said calcium salt is calcium citrate.

26. The method of claim 21, further comprising the step of making said solution with a pH of more than 3 and less than 6, and said calcium salt is calcium citrate.

27. The method of claim 21, further comprising the step of making said solution with a pH of more than 5 and less than 6, and said calcium salt is calcium citrate.

28. The method of claim 21, further comprising the step of making said solution with a pH of less than 6, said acidic buffer is citric acid, and said calcium salt is calcium citrate.

29. The method of claim 21, further comprising the step of making said solution with a pH of more than 3 and less than 6, said acidic buffer is citric acid, and said calcium salt is calcium citrate.

30. The method of claim 21, further comprising the step of making said solution with a pH of more than 5 and less than 6, said acidic buffer is citric acid, and said calcium salt is calcium citrate.

31. The method of claim 21, further comprising the step of making said solution an injectable.

32. The method of claim 21, further comprising the step of making said solution in a form allowing said solution to be delivered via an oral transmucosal method which is not an injectable.

33. The method of claim 32, further comprising the step of making said transmucosal method a gel.

34. The method of claim 33, wherein said gel transmucosal method further comprises a gelling agent, an excipient, and a flavor.

35. The method of claim 33, wherein said gel transmucosal method further comprises a gelling agent at 1 to 10% by weight, an excipient at 1 to 20% by weight, said acidic buffer at 1 to 20% by weight, and total to 100% by weight with flavor and said deionized water.

36. The method of claim 33, wherein said gel has glycol as a gelling agent and/or excipient.

37. The method of claim 36, wherein said gel has polyethylene glycol and propylene glycol as a gelling agent and/or excipient.

38. The method of claim 34, wherein said flavor to enhance taste comprises a simple sugar, or a non-sugar sweetener.

39. The method of claim 32, further comprising the step of making said transmucosal method a lozenge.

40. The method of claim 32, further comprising the step of making said transmucosal method a starched based dissolving film.
